SEQUENCE LISTING (8 sequences)

SEQ ID NO: 1 - protein, 1403 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCMRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIAKVAISGGFQKLENLKLSINHKITEEGYRNFFQALDNMPNLQELDISRHFTECIKAQATTVKSLSQCVLRLPRLIRLNMLSWLLDADDIALLNVMKERHPQSKYLTILQKWILPFSPIIQK

SEQ ID NO: 2 - DNA, 5984 bp, Homo sapiens
  CDS (292)..(4500)
acaaaaggtcctgtgctcacctgggacccttctggacgttgccctgtgttcctcttcgcctgcctgttcatctacgacgaaccccgggtattgaccccagacaacaatgccacttcatattggggacttcgtctgggattccaaggtgcattcattgcaaagttccttaaatattttctcactgcttcctactaaaggacggacagagcatttgttcttcagccacatactttccttccactggccagcattctcctctattagactagaactgtggataaacctcagaaaatggccacccagcagaaagcctctgacgagaggatctcccagtttgatcacaatttgctgccagagctgtctgctcttctgggcctagatgcagttcagttggcaaaggaactagaagaagaggagcagaaggagcgagcaaaaatgcagaaaggctacaactctcaaatgcgcagtgaagcaaaaaggttaaagacttttgtgacttatgagccgtacagctcatggataccacaggagatggcggccgctgggttttacttcactggggtaaaatctgggattcagtgcttctgctgtagcctaatcctctttggtgccggcctcacgagactccccatagaagaccacaagaggtttcatccagattgtgggttccttttgaacaaggatgttggtaacattgccaagtacgacataagggtgaagaatctgaagagcaggctgagaggaggtaaaatgaggtaccaagaagaggaggctagacttgcatccttcaggaactggccattttatgtccaagggatatccccttgtgtgctctcagaggctggctttgtctttacaggtaaacaggacacggtacagtgtttttcctgtggtggatgtttaggaaattgggaagaaggagatgatccttggaaggaacatgccaaatggttccccaaatgtgaatttcttcggagtaagaaatcctcagaggaaattacccagtatattcaaagctacaagggatttgttgacataacgggagaacattttgtgaattcctgggtccagagagaattacctatggcatcagcttattgcaatgacagcatctttgcttacgaagaactacggctggactcttttaaggactggccccgggaatcagctgtgggagttgcagcactggccaaagcaggtcttttctacacaggtataaaggacatcgtccagtgcttttcctgtggagggtgtttagagaaatggcaggaaggtgatgacccattagacgatcacaccagatgttttcccaattgtccatttctccaaaatatgaagtcctctgcggaagtgactccagaccttcagagccgtggtgaactttgtgaattactggaaaccacaagtgaaagcaatcttgaagattcaatagcagttggtcctatagtgccagaaatggcacagggtgaagcccagtggtttcaagaggcaaagaatctgaatgagcagctgagagcagcttataccagcgccagtttccgccacatgtctttgcttgatatctcttccgatctggccacggaccacttgctgggctgtgatctgtctattgcttcaaaacacatcagcaaacctgtgcaagaacctctggtgctgcctgaggtctttggcaacttgaactctgtcatgtgtgtggagggtgaagctggaagtggaaagacggtcctcctgaagaaaatagcttttctgtgggcatctggatgctgtcccctgttaaacaggttccagctggttttctacctctcccttagttccaccagaccagacgaggggctggccagtatcatctgtgaccagctcctagagaaagaaggatctgttactgaaatgtgcatgaggaacattatccagcagttaaagaatcaggtcttattccttttagatgactacaaagaaatatgttcaatccctcaagtcataggaaaactgattcaaaaaaaccacttatcccggacctgcctattgattgctgtccgtacaaacagggccagggacatccgccgatacctagagaccattctagagatcaaagcatttcccttttataatactgtctgtatattacggaagctcttttcacataatatgactcgtctgcgaaagtttatggtttactttggaaagaaccaaagtttgcagaagatacagaaaactcctctctttgtggcggcgatctgtgctcattggtttcagtatccttttgacccatcctttgatgatgtggctgttttcaagtcctatatggaacgcctttccttaaggaacaaagcgacagctgaaattctcaaagcaactgtgtcctcctgtggtgagctggccttgaaagggtttttttcatgttgctttgagtttaatgatgatgatctcgcagaagcaggggttgatgaagatgaagatctaaccatgtgcttgatgagcaaatttacagcccagagactaagaccattctaccggtttttaagtcctgccttccaagaatttcttgcggggatgaggctgattgaactcctggattcagataggcaggaacatcaagatttgggactgtatcatttgaaacaaatcaactcacccatgatgactgtaagcgcctacaacaattttttgaactatgtctccagcctcccttcaacaaaagcagggcccaaaattgtgtctcatttgctccatttagtggataacaaagagtcattggagaatatatctgaaaatgatgactacttaaagcaccagccagaaatttcactgcagatgcagttacttaggggattgtggcaaatttgtccacaagcttacttttcaatggtttcagaacatttactggttcttgccctgaaaactgcttatcaaagcaacactgttgctgcgtgttctccatttgttttgcaattccttcaagggagaacactgactttgggtgcgcttaacttacagtactttttcgaccacccagaaagcttgtcattgttgaggagcatccacttcccaatacgaggaaataagacatcacccagagcacatttttcagttctggaaacatgttttgacaaatcacaggtgccaactatagatcaggactatgcttctgcctttgaacctatgaatgaatgggagcgaaatttagctgaaaaagaggataatgtaaagagctatatggatatgcagcgcagggcatcaccagaccttagtactggctattggaaactttctccaaagcagtacaagattccctgtctagaagtcgatgtgaatgatattgatgttgtaggccaggatatgcttgagattctaatgacagttttctcagcttcacagcgcatcgaactccatttaaaccacagcagaggctttatagaaagcatccgcccagctcttgagctgtctaaggcctctgtcaccaagtgctccataagcaagttggaactcagcgcagccgaacaggaactgcttctcaccctgccttccctggaatctcttgaagtctcagggacaatccagtcacaagaccaaatctttcctaatctggataagttcctgtgcctgaaagaactgtctgtggatctggagggcaatataaatgttttttcagtcattcctgaagaatttccaaacttccaccatatggagaaattattgatccaaatttcagctgagtatgatccttccaaactagtaaaattaattcaaaattctccaaaccttcatgttttccatctgaagtgtaacttcttttcggattttgggtctctcatgactatgcttgtttcctgtaagaaactcacagaaattaagttttcggattcattttttcaagccgtcccatttgttgccagtttgccaaattttatttctctgaagatattaaatcttgaaggccagcaatttcctgatgaggaaacatcagaaaaatttgcctacattttaggttctcttagtaacctggaagaattgatccttcctactggggatggaatttatcgagtggccaaactgatcatccagcagtgtcagcagcttcattgtctccgagtcctctcatttttcaagactttgaatgatgacagcgtggtggaaattgccaaagtagcaatcagtggaggtttccagaaacttgagaacctaaagctttcaatcaatcacaagattacagaggaaggatacagaaatttctttcaagcactggacaacatgccaaacttgcaggagttggacatctccaggcatttcacagagtgtatcaaagctcaggccacaacagtcaagtctttgagtcaatgtgtgttacgactaccaaggctcattagactgaacatgttaagttggctcttggatgcagatgatattgcattgcttaatgtcatgaaagaaagacatcctcaatctaagtacttaactattctccagaaatggatactgccgttctctccaatcattcagaaataaaagattcagctaaaaactgctgaatcaataatttgtcttggggcatattgaggatgtaaaaaaagttgttgattaatgctaaaaaccaaattatccaaaattattttattaaatattgcatacaaaagaaaatgtgtaaggcttgctaaaaaacaaaacaaaacaaaacacagtcctgcatactcaccaccaagctcaagaaataaatcatcaccaatacctttgaggtccctgagtaatccaccccagctaaaggcaaacccttcaatcaagtttatacagcaaaccctccattgtccatggtcaacagggaaggggttggggacaggtctgccaatctatctaaaagccacaatatggaagaagtattcaatttatataataaatggctaacttaacggttgaatcactttcatacatggatgaaacgggtttaacacaggatccacatgaatcttctgtgggccaaaatatgttccttaatccttgtagaacctgtcttctatattgaactagctttggtacagtagagttaacttactttccatttatccactgccaatataaagaggaaacaggggttagggaaaaatgacttcattccagaggcttctcagagttcaacatatgctataatttagaattttcttatgaatccactctacttgggtagaaaatattttatctctagtgattgcatattatttccatatcatagtatttcatagtattatatttgatatgagtgtctatatcaatgtcagtgtccagaatttcgttcctaccagttgagtagttttctgaacggccagaagaccattcgaaattcatgatactactataagttggtaaacaaccatacttttatcctcatttttattctcactaagaaaaaagtcaactcccctccccttgcccaagtatgaaatatagggacagtatgtatggtgtggtctcatttgtttagaaaaccacttatgactgggtgcggtggctcacacctgtaatcccagcactttgggaggctgaggcgggcgaatcatttgaggtgaggagttcgagaccggcctggccagcatggtgaaaccccatttttgctaaaggtacaaaaattagccaggtgtggtggcacatgcctgtggtcccagccactggggcggctgagacgcaggacttgcttgaacccgggaggcagaggttgcagtgagccgagatggcgccactgcattccagcctgggcaacagagcaagaccctgtctgtttcaaaacaaaaaacaaaaccacttatattgctagctacattaagaatttctgaatatgttactgagcttgcttgtggtaaccatttataatatcagaaagtatatgtacaccaaaacatgttgaacatccatgttgtacaactgaaatataaataattttgtcaattatacctaaataaaactggaaaaaaaaaaaaaaaaaaaaaaaaaaaaaaaaa

SEQ ID NO: 3 - protein, 1295 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCMRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIGELVFQLAWKPVV

SEQ ID NO: 4 - DNA, 5366 bp, Homo sapiens
  CDS (292)..(4176)
acaaaaggtcctgtgctcacctgggacccttctggacgttgccctgtgttcctcttcgcctgcctgttcatctacgacgaaccccgggtattgaccccagacaacaatgccacttcatattggggacttcgtctgggattccaaggtgcattcattgcaaagttccttaaatattttctcactgcttcctactaaaggacggacagagcatttgttcttcagccacatactttccttccactggccagcattctcctctattagactagaactgtggataaacctcagaaaatggccacccagcagaaagcctctgacgagaggatctcccagtttgatcacaatttgctgccagagctgtctgctcttctgggcctagatgcagttcagttggcaaaggaactagaagaagaggagcagaaggagcgagcaaaaatgcagaaaggctacaactctcaaatgcgcagtgaagcaaaaaggttaaagacttttgtgacttatgagccgtacagctcatggataccacaggagatggcggccgctgggttttacttcactggggtaaaatctgggattcagtgcttctgctgtagcctaatcctctttggtgccggcctcacgagactccccatagaagaccacaagaggtttcatccagattgtgggttccttttgaacaaggatgttggtaacattgccaagtacgacataagggtgaagaatctgaagagcaggctgagaggaggtaaaatgaggtaccaagaagaggaggctagacttgcatccttcaggaactggccattttatgtccaagggatatccccttgtgtgctctcagaggctggctttgtctttacaggtaaacaggacacggtacagtgtttttcctgtggtggatgtttaggaaattgggaagaaggagatgatccttggaaggaacatgccaaatggttccccaaatgtgaatttcttcggagtaagaaatcctcagaggaaattacccagtatattcaaagctacaagggatttgttgacataacgggagaacattttgtgaattcctgggtccagagagaattacctatggcatcagcttattgcaatgacagcatctttgcttacgaagaactacggctggactcttttaaggactggccccgggaatcagctgtgggagttgcagcactggccaaagcaggtcttttctacacaggtataaaggacatcgtccagtgcttttcctgtggagggtgtttagagaaatggcaggaaggtgatgacccattagacgatcacaccagatgttttcccaattgtccatttctccaaaatatgaagtcctctgcggaagtgactccagaccttcagagccgtggtgaactttgtgaattactggaaaccacaagtgaaagcaatcttgaagattcaatagcagttggtcctatagtgccagaaatggcacagggtgaagcccagtggtttcaagaggcaaagaatctgaatgagcagctgagagcagcttataccagcgccagtttccgccacatgtctttgcttgatatctcttccgatctggccacggaccacttgctgggctgtgatctgtctattgcttcaaaacacatcagcaaacctgtgcaagaacctctggtgctgcctgaggtctttggcaacttgaactctgtcatgtgtgtggagggtgaagctggaagtggaaagacggtcctcctgaagaaaatagcttttctgtgggcatctggatgctgtcccctgttaaacaggttccagctggttttctacctctcccttagttccaccagaccagacgaggggctggccagtatcatctgtgaccagctcctagagaaagaaggatctgttactgaaatgtgcatgaggaacattatccagcagttaaagaatcaggtcttattccttttagatgactacaaagaaatatgttcaatccctcaagtcataggaaaactgattcaaaaaaaccacttatcccggacctgcctattgattgctgtccgtacaaacagggccagggacatccgccgatacctagagaccattctagagatcaaagcatttcccttttataatactgtctgtatattacggaagctcttttcacataatatgactcgtctgcgaaagtttatggtttactttggaaagaaccaaagtttgcagaagatacagaaaactcctctctttgtggcggcgatctgtgctcattggtttcagtatccttttgacccatcctttgatgatgtggctgttttcaagtcctatatggaacgcctttccttaaggaacaaagcgacagctgaaattctcaaagcaactgtgtcctcctgtggtgagctggccttgaaagggtttttttcatgttgctttgagtttaatgatgatgatctcgcagaagcaggggttgatgaagatgaagatctaaccatgtgcttgatgagcaaatttacagcccagagactaagaccattctaccggtttttaagtcctgccttccaagaatttcttgcggggatgaggctgattgaactcctggattcagataggcaggaacatcaagatttgggactgtatcatttgaaacaaatcaactcacccatgatgactgtaagcgcctacaacaattttttgaactatgtctccagcctcccttcaacaaaagcagggcccaaaattgtgtctcatttgctccatttagtggataacaaagagtcattggagaatatatctgaaaatgatgactacttaaagcaccagccagaaatttcactgcagatgcagttacttaggggattgtggcaaatttgtccacaagcttacttttcaatggtttcagaacatttactggttcttgccctgaaaactgcttatcaaagcaacactgttgctgcgtgttctccatttgttttgcaattccttcaagggagaacactgactttgggtgcgcttaacttacagtactttttcgaccacccagaaagcttgtcattgttgaggagcatccacttcccaatacgaggaaataagacatcacccagagcacatttttcagttctggaaacatgttttgacaaatcacaggtgccaactatagatcaggactatgcttctgcctttgaacctatgaatgaatgggagcgaaatttagctgaaaaagaggataatgtaaagagctatatggatatgcagcgcagggcatcaccagaccttagtactggctattggaaactttctccaaagcagtacaagattccctgtctagaagtcgatgtgaatgatattgatgttgtaggccaggatatgcttgagattctaatgacagttttctcagcttcacagcgcatcgaactccatttaaaccacagcagaggctttatagaaagcatccgcccagctcttgagctgtctaaggcctctgtcaccaagtgctccataagcaagttggaactcagcgcagccgaacaggaactgcttctcaccctgccttccctggaatctcttgaagtctcagggacaatccagtcacaagaccaaatctttcctaatctggataagttcctgtgcctgaaagaactgtctgtggatctggagggcaatataaatgttttttcagtcattcctgaagaatttccaaacttccaccatatggagaaattattgatccaaatttcagctgagtatgatccttccaaactagtaaaattaattcaaaattctccaaaccttcatgttttccatctgaagtgtaacttcttttcggattttgggtctctcatgactatgcttgtttcctgtaagaaactcacagaaattaagttttcggattcattttttcaagccgtcccatttgttgccagtttgccaaattttatttctctgaagatattaaatcttgaaggccagcaatttcctgatgaggaaacatcagaaaaatttgcctacattttaggttctcttagtaacctggaagaattgatccttcctactggggatggaatttatcgagtggccaaactgatcatccagcagtgtcagcagcttcattgtctccgagtcctctcatttttcaagactttgaatgatgacagcgtggtggaaattggtgagctagtgtttcagcttgcatggaagccagtggtatagccaagctttctgctgcaacatgtctatgtaaacatttgcccctctagaaattttcaacccgcttcctcattttcactatcatactgttccttctagtgtccttctgtggatttaggcgcattctggtcagatttggaagtacaaaaaggtctcccatttgtggatatacaagccctcaaatctgcgttcttgccacctggtgttttagacacctggccacatactctcctaagtactcctttttaaaactgaagatgaatatacacacagaaaagtacaaaaatcatgtgtactgctcactgaattttattttcttattttcttcttttttttttttttgagacagagtttcgctcgtgttgcccaggctggagtacaatggcacgatctcgggtcactgcaaactctgcctcctgggttcaagcgattctcctgcctcagcctcccaagtagctaggattacaggtgaacgccaccacacctggctaattttgtatttttagtaaacacagggtttcaccatgttggccaggctagtctcgaactcctgacctcaagtgagccacagtgcctggcctgaggaactgagatttctgtcgagacctgaagggagaatggcccaggcatagttggtagaggaggaattgagacatcatttcaaacagaggtaatcacttgtgtcatagcctggagttaaagagaaccagatatatttgaagaacttgggggaaaaaaaggaatgtctggagcaagaggcaggagtgagttgtgagaagaagactggagaggaaagtaaaagcccaattggagaggctttgtcgggtgtgttacaagggctggatctcattttcttactgctcagcactgttattttacgttatttaaaacagctgggagcggtggctcaagcttgtaatcccagcactttgggaggccgaggcggatggatcacgaggtcaggagatcgagaccatcctggctaacatggtgaaaccccgtctctactaaaaatacaaaaaattagccaggcgtgatggcgggcacctgtagtcccagctactcgggaggctgaggcaggagaatggtgtgaacccgggaggtggagcttgaagtgagccaagatcatgccactgcactccagcctgggcaacagaacgagactccgtctcaaaaaaaaaaaacaaaaa

SEQ ID NO: 5 - DNA, 31 bp, Artificial Sequence
  Artificial Sequence Synthesized oligonucleotide
gaggaattcctacattttaggttctcttagt

SEQ ID NO: 6 - DNA, 28 bp, Artificial Sequence
  Artificial Sequence Synthesized oligonucleotide
gaggaattcaatttccaccacgctgtca

SEQ ID NO: 7 - DNA, 20 bp, Artificial Sequence
  Artificial Sequence Synthesized oligonucleotide
aattaaccctcactaaaggg

SEQ ID NO: 8 - DNA, 24 bp, Artificial Sequence
  Artificial Sequence Synthesized oligonucleotide
cattatgctgagtgtgatatcccg

(19) United States
(12) Patent Application Publication
(10) Pub. No.: US 2005/0100997 A1
(43) Pub. Date: May 12, 2005

(54) Apoptosis inhibitory protein, gene encoding the protein and cDNA thereof

(76) Inventors: Joh-E Ikeda, Tokyo (JP); Kenji Yamamoto, Kanagawa (JP)

Correspondence Address: WENDEROTH, LIND & PONACK, L.L.P., 2033 K STREET N. W., SUITE 800, WASHINGTON, DC, 20006-1021, US

(21) Appl. No.: 10/628,324

(22) Filed: Jul. 29, 2003

Related U.S. Application Data
(62) Division of application No. 09/705,872, filed on Nov. 6, 2000, now Pat. No. 6,617,429.
Application No. 09/705,872 is a continuation-in-part of application No. 09/239,797, filed on Jan. 29, 1999, now abandoned.

(51) Int. Cl. C12N 9/99; C07H 21/04; C07K 16/40

(52) U.S. Cl. 435/184; 435/69.2; 435/320.1; 435/325; 530/388.26; 536/23.2

(57) ABSTRACT

Novel apoptosis inhibitory proteins, a gene encoding the proteins and the cDNAs thereof are provided. More particularly, human apoptosis inhibitory proteins comprising the amino acid sequence of SQ ID No.1 or 3, a human gene encoding the human apoptosis inhibitory proteins and the cDNAs of the human gene comprising at least the nucleotide sequence for the coding region of SQ ID No.2 or 4, are provided.

### FIELD OF THE INVENTION

The present invention relates to a human apoptosis inhibitory protein, and a gene encoding the protein and the cDNA thereof. More specifically, the present invention relates to the genetic materials which are useful for the elucidation of the onset mechanism of various apoptic diseases such as human spinal muscular atropy, the diagnosis of the risk of the onset thereof, and the prevention of the onset thereof. In addition, the materials are useful for the development of clinical techniques and pharmaceutical agents for the amelioration and therapeutic treatment of the diseases.

### PRIOR ART

Apoptosis is a programmed cellular death, involving observed phenomena such as the loss of cellular contact with surrounding cells, cytoplasmic condensation, chromatin condensation and nuclear condensation with relation to endonuclease activity, nuclear fragmentation, membrane-enveloped spherical microbodies, the phagocytosis of spherical microbodies with adjacent macrophages or epithelial cells, or the fragmentation of the DNA nucleosome unit into DNAs of 180 to 200 bp due to endonuclease activity. It is suggested that apoptosis is a phagocytic mechanism for the final fragment of an apoptic somatic cell under such observed phenomena by adjacent cells (see for example Immunology Today 7: 115-119, 1986; Science 245:301-305, 1989).

As an apoptosis inhibitory gene, for example, gene bcl-2 has been known. The gene bcl-2, one of oncogenes discovered in 1985 in alveolar B cytoma, is highly expressed in the immune system and nervous system, and it is believed that the expression product of the gene serves to maintain the homeostasis of the human immune functions and neuronal functions, by inhibiting the apoptosis of the cells involved. Additionally because the bcl-2 is expressed in a diversified range in fetuses in particular, the gene is believed to play a significant role in morphological formation during ontogenesis.

Meanwhile, the present inventors have isolated the gene of a neuronal apoptosis inhibitory protein (NAIP) from the human chromosome 5q13.1 region as an etiological gene of a familial hereditary disease spinal muscular atropy (SMA) (Roy et al., Cell 80: 167-178, 1995), and have filed a patent application (PCT/CA95/00581). More specifically, it is supposed that the mutation of the NAIP gene or the decrease of the copy number thereof might cause the apoptosis of spinal neuron, which is an etiology of the SMA onset. It is apparently demonstrated that by introducing the NAIP gene into various cultured cells to give apoptosis-inducing stimulation to the cells, the death of the cells is inhibited (Liston et al., Nature 379: 349-353,1996), which indicates that NAIP plays a role of an apoptosis inhibitory factor for not only neuronal cells but also overall somatic cells.

### SUMMARY OF THE INVENTION

The present inventors have further promoted the analysis of the NAIP gene, and they have successfully achieved to clone the full length of cDNA of NAIP gene and to identify the protein encoded in the cDNA.

It is an object of the present invention to provide the cDNA of NAIP gene thus found by the present inventors, genetic materials with relation to the cDNA and the expression products thereof and the like in industrially applicable forms.

An invention provided by the present application is a human apoptosis inhibitory protein which comprises the amino acid sequence of SQ ID No:1, or an amino acid sequence with deletion, substitution or addition of a single or plural amino acids in SQ ID No:1.

Another invention is a human apoptosis inhibitory protein comprising the amino acid sequence of SQ ID No:3, or an amino acid sequence with deletion, substitution or addition of a single or plural amino acids in SQ ID No:3.

Other inventions are a human gene encoding the human apoptosis inhibitory proteins, cDNAs of said human gene which comprises at least the nucleotide sequence for the coding region of SQ ID No:2 or NO:4.

Still additionally, inventions of this application are an antibody against the human apoptosis inhibitory proteins, a non-human animal gene to which the above cDNAs are hybridized, recombinant vector carrying the cDNAs or a partial sequence thereof, a DNA probe comprising a partial sequence of the cDNAs, and a set of PCR primer corresponding to partial sequences of the cDNAs.

The present inventions will now be described below in more detail with reference to embodiments.

### BRIEF DESCRIPTION OF DRAWING

FIG. 1 schematically depicts the individual 3′-terminal structures of the conventionally known apoptosis inhibitory gene NAIP_{S} and the inventive genes NAIP_{M} and NAIP_{L}.

### DETAILED DESCRIPTION OF THE INVENTION

The human apoptosis inhibitory protein of the present invention is a human protein comprising the amino acid sequence of SQ ID No.1 or 3. A peptide (with 5 amino acid residues or more) consisting of any partial amino acid sequence of the amino acid sequence of SQ ID No.1 or 3 is included in the scope of this protein. Such peptide may be used as an antigen to prepare an antibody, for example. Furthermore, the protein of the present invention includes fusion proteins with other proteins (for example, fluorescent proteins).

According to known methods, the protein of the present invention may be isolated from human organs or cell lines. When intending to use the protein as a peptide, the protein may be prepared on the basis of the amino acid sequences provided by the present invention by chemical synthesis. Otherwise, the protein may be obtained through in vitro transcription or a recombinant DNA technique by using a cDNA fragment provided by the present invention. In order to obtain the protein by the recombinant DNA technique, for example, the protein of the present invention may be expressed at a large scale from a host cell (Escherichia coli, Bacillus subtilis, yeast, animal or plant cells, etc.) which has been transformed by a recombinant vector prepared by inserting the cDNA fragment of the present invention in an appropriate expression vector. For expressing the protein in a microorganism such as Escherichia coli, more specifically, the cDNA of the present invention is inserted within an expression vector having an origin suitable for the microorganism, a promoter sequence, a ribosome-binding site, DNA cloning sites, a terminator sequence and the like to prepare an expression vector, which is used to transform a host cell and thereafter culture the resulting transformant, whereby a protein encoded by the cDNA can be produced in the microorganism at a large scale. Otherwise, the protein may be expressed in the form of a fused protein with other proteins. By hydrolyzing the resulting fused protein with an appropriate protease, a protein part encoded by the cDNA may be recovered. For intending to allow the protein of the present invention to be expressed and secreted in an animal cell, alternatively, the cDNA fragment is inserted within an animal cell expression vector with an animal cell promoter, a splicing region, a poly(A) additional site, and the like, the protein of the present invention may be expressed in the animal cell.

The gene of the present invention is derived from humans and other mammals and encodes the protein, and can be isolated from the known genomic libraries by using the cDNA of the present invention or a partial sequence thereof as the probe.

The cDNA of the present invention comprises the nucleotide sequence of SQ ID No.2 or 4. The cDNAs of the nucleotide sequences of SQ ID Nos.2 and 4 encode the proteins of the amino acid sequences of SQ ID Nos. 1 and 3, respectively.

Because the protein of the present invention is expressed in any human tissue, a clone identical to the cDNA of the present invention may readily be recovered by screening human cDNA libraries by using an oligonucleotide probe synthesized on the basis of the nucleotide sequence of the cDNA of SQ ID No.2 or 4. Otherwise, the objective cDNA may be synthesized by polymerase chain reaction (PCR) by using such oligonucleotides as primers. Generally, it is frequently observed that human genes have polymorphism due to differences of individual nucleotide. Thus, cDNAs in which the addition and deletion of a single or plural nucleotides and/or the substitution with a single or plural nucleotides occur in SQ ID No.2 or 4 are also encompassed within the scope of the present invention. Similarly, proteins in which the addition and deletion of a single or plural amino acid residues and/or the substitution with a single or plural amino acid residues occur due to such modification are also encompassed within the scope of the present invention, as long as the proteins have the activities of the protein with the amino acid sequence of SQ ID No.1 or 3.

Additionally, the partial sequence of the cDNA of the present invention is a continuous sequence of 10 bp or more in the nucleotide sequence of SQ ID No.2 or 4, and DNA fragments (sense chain and antisense chain) comprising such continuous sequence are also encompassed within the scope of the present invention. These DNA fragments may be used as probes for genetic diagnosis, for example.

Furthermore, the antibody of the present invention may be prepared in the form of a polyclonal antibody or monoclonal antibody, by known methods by using the protein described above of itself or a partial peptide thereof as an antigen.

The present invention will now be described more specifically in more detail in examples, but the invention is not limited to the following examples.

### EXAMPLES

### Example 1

### Screening of cDNA Library

Exxon 16 of the NAIP gene was PCR amplified by using the oligonucleotides of SQ ID Nos.5 and 6 as primers. PCR conditions were as follows; 94° C. for 15 seconds, 56° C. for 30 seconds and 72° C. for one minute.

By using the resulting PCR product, then, the cDNA library of human fetal brain (NA 937227; Stratagene) was screened. As a result, eight clones with overlaps with the NAIP gene were identified.

As a result of the sequence analysis, the eight cDNA clones were separated into seven clones having the same coding region at the 3′ termini and one clone comprising a shorter DNA fragment than those of the seven clones. Based on the length of the DNA fragments, furthermore, it was identified that the genes encoding these clones were longer DNA molecules than the NAIP gene previously reported.

For convenience, hereinafter, the conventionally known NAIP gene is referred to as NAIP_{S}; the gene encoding the longer cDNA thus screened is referred to as NAIP_{L}; and the shorter gene is referred to as NAIP_{M}.

### Example 2

### Sequencing of the cDNAs

The nucleotide sequences of the cDNA clones identified in Example 1 were determined. By using the sequences determined by using the oligonucleotides of SQ ID Nos.7 and 8 as primary primers, additional primers were sequentially prepared, to determine the full sequences of the cDNAs by the walking method.

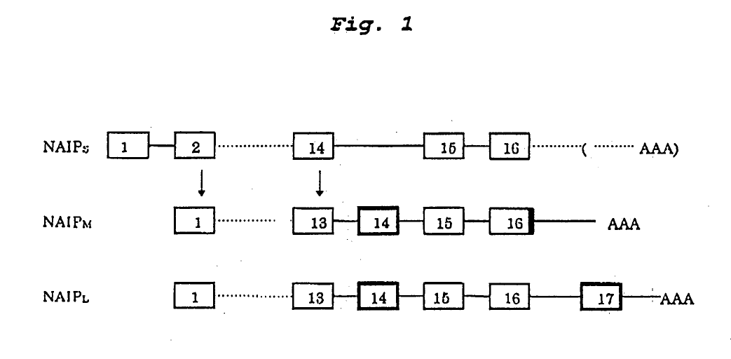

Consequently, it is confirmed that the conventionally known exons of NAIP_{S} (upper column, FIG. 1) is inaccurate. NAIP_{M} and NAIP_{L} do not have exon 1 of NAIP_{S} and have a new exon (153 bp) between the exons 14 and 15 of the NAIP_{S} (middle and lower columns, FIG. 1). Additionally, it is confirmed that NAIP_{L} have an additional exon at the 3′ terminus of the NAIP_{M} (lower columns, FIG. 1).

In other words, the NAIP is expressed in two splice variant forms, NAIP_{M} with exons 1 to 16 and NAIP_{L} with exons 1 to 17. In more detail, NAIP_{M} has the novel exon 14 and additionally contains extra 39 bp at the 3′ terminus of the exon 16, while the cDNA thereof has the nucleotide sequence of SQ ID No.4 and encodes the protein of the amino acid sequence of SQ ID No.3. On the other hand, NAIP_{L} contains exon 17 of 363 bp in addition to the exon 14, while the cDNA thereof has the nucleotide sequence of SQ ID No.2 and encodes the protein of the amino acid sequence of SQ ID No. 1.

Based on the aforementioned results, it is verified that the apoptosis inhibitory genes NAIP_{M} and NAIP_{L} of the present invention are novel genes, apparently different from the conventionally known gene NAIP_{S}; and that the apoptosis inhibitory proteins encoded by these genes are novel proteins.

### Example 3

### Expression of protein in Escherichia coli

A translated region was PCR amplified by using an NAIP_{L}-containing clone isolated in Example 1 as template. The resulting PCR product was inserted into an expression vector for Escherichia coli, and after confirming the nucleotide sequence of the insert, the host Escherichia coli was transformed with the vector. The transformant was cultured in an LB culture medium at 37° C. for 5 hours, followed by addition of IPTG to a final concentration of 0.4 mM and subsequent additional culturing at 37° C. for 2.5 hours. The bacteria were centrifuged and isolated, and were then dissolved in a dissolving solution, and the resulting solution was once frozen at −80° C. and thawed, for ultrasonic disruption. The solution in disruption was centrifuged, and from the resulting supernatant was isolated and purified a protein, which was recovered as the apoptosis inhibitory protein (SQ ID No.1) of the present invention.

### Example 4

### Preparation of Antibody

A rabbit was immunized with the protein obtained in Example 3 as an antigen, to prepare an anti-serum. From the antiserum was first removed a 40%-saturated ammonium sulfate precipitate fraction on a GST affinity column. The pass-through fraction was further purified on an antigen column GST-HP10345.

As has been described above, the novel apoptosis inhibitory proteins, the gene encoding the proteins and the cDNAs thereof are provided in accordance with the present invention, whereby the elucidation of the onset mechanism of various apoptic diseases primarily including human spinal muscular atropy, the diagnosis of the risk of the onset thereof, the prevention of the onset thereof and the amelioration of the diseased conditions, and the development of clinical techniques and pharmaceutical agents for the therapeutic treatment, can be attained.

1-16. (canceled)

17. A polynucleotide encoding a polypeptide comprising (a) the amino acid sequence of SEQ ID NO: 1 or 3, or (b) the amino acid sequence of SEQ ID NO: 1 or 3 having a deletion, substitution and/or addition of one or more amino acid(s).

18. The polynucleotide according to claim 17, which is a DNA or cDNA.

19. The polynucleotide according to claim 18, wherein the DNA or cDNA is human DNA or cDNA.

20. The polynucleotide according to claim 17, which comprises the nucleotide sequence of SEQ ID No. 2 or 4.

21. The polynucleotide according to claim 20, which is a DNA or cDNA.

22. The polynucleotide according to claim 21, wherein the DNA or cDNA is human DNA or cDNA.

23. An antibody against a polypeptide encoded by the polynucleotide of claim 17.

24. A polynucleotide which hybridizes to the polynucleotide of claim 20 or a partial sequence thereof.

25. The polynucleotide according to claim 24, which is a DNA or cDNA.

26. The polynucleotide according to claim 25, wherein the DNA or cDNA is nonhuman animal DNA or cDNA.

27. A recombinant vector comprising the polynucleotide of claim 17 or a partial sequence thereof.

28. A recombinant vector comprising the polynucleotide of claim 20 or a partial sequence thereof.

29. A DNA probe comprising a partial sequence of the polynucleotide of claim 17.

30. A DNA probe comprising a partial sequence of the polynucleotide of claim 20.

31. A set of PCR primer comprising partial sequences of the polynucleotide of claim 17.

32. A set of PCR primer comprising partial sequences of the polynucleotide of claim 20.

33. A PCR primer comprising a partial sequence of the polynucleotide of claim 17.
